(19) United States
(12) Patent Application Publication
(10) Pub. No.: US 2026/0097240 A1
(43) Pub. Date: Apr. 9, 2026

(54) ELASTIC MESH SLEEVE DEVICE FOR ULTRASONIC TREATMENT

(71) Applicant: Zhijun PENG, Foshan (CN)

(72) Inventor: Zhijun PENG, Foshan (CN)

(21) Appl. No.: 18/991,754

(22) Filed: Dec. 23, 2024

(30) Foreign Application Priority Data
Oct. 6, 2024 (CN) 2024113876845

(51) Int. Cl. A61N 7/02

(52) U.S. Cl. CPC A61N 7/02

(57) ABSTRACT

An elastic mesh sleeve device for ultrasonic treatment, including an elastic biomaterial mesh sleeve, and a micro ultrasonic vibration module having a housing, a housing cover, an ultrasonic vibration assembly, and a driving control assembly. Due to the biomaterials that the elastic mesh sleeve device is made of, it can be safely used inside a human body to sleeve directly onto a site where treatment is needed so as to directly perform ultrasonic wave therapy to the site.

### BACKGROUND OF THE INVENTION

The present invention relates to the field of ultrasonic therapy devices for a human body, and in particular to an elastic mesh sleeve device for ultrasonic treatment.

An ultrasonic therapy device makes use of ultrasonic waves as the means for therapeutic treatment, wherein the ultrasonic waves outside the body are focused on a body part inside which pathological tissues are present; due to the ultrasonic waves, a temperature of the pathological tissues inside the body increases quickly, and a biological impact is resulted under mechanical reactions, thermal penetration and cavitation, thereby destroying the structures of the cells of the pathological tissues and causing the cells of the pathological tissues to wither. In addition, ultrasonic waves can promote blood circulation, accelerate metabolism, regenerate healthy tissues, and achieve a purpose of treating and curing the pathological tissues.

Prior art CN111544782A (application number 202010504927.4) titled “real-time positioning low-energy ultrasonic therapy device” has disclosed one of the ultrasonic therapy devices in the prior art; said prior art ultrasonic therapys mainly a treatment station and an ultrasonic irradiation probe; the treatment station ultrasonic therapys a support device, a control panel, an image display screen, and an ultrasonic generation device; the ultrasonic irradiation probe is connected to the ultrasonic generation device. During treatment, a medical personnel places the ultrasonic irradiation probe on the skin of a patient, and then the ultrasonic irradiation probe will radiate ultrasonic waves towards the patient and perform ultrasonic therapy on the body part to be treated. However, in practical application, the ultrasonic therapy device has the following deficiencies:

Firstly, this kind of ultrasonic therapy device is used externally outside the patient's body, therefore, the ultrasonic waves can only reach the site containing the pathological tissues after passing through the skin of the patient, and as a result, the ultrasonic waves cannot directly reach the site in concern. Moreover, the skin tissues can absorb part of the radiated ultrasonic energy, therefore the radiated ultrasonic energy that actually reaches the site is weakened. This reduces the destructive power to the cells of the pathological tissues and thus leads to slow therapeutic effect. If the intensity of the ultrasonic energy is increased, more significant side effects like damages to the skin tissues and cells will be resulted in spite of the fact that the pathological tissues can be killed under more intense ultrasonic energy; therefore, this approach of increasing the intensity of ultrasonic energy is still not preferred.

Secondly, only a professional medical personnel in a hospital knows how to operate the ultrasonic therapy device, and so the patient has to travel to the hospital frequently to receive regular treatment, thereby leading to extremely high costs in terms of both time and money.

Therefore, the applicant believes that it is highly necessary to design an elastic mesh sleeve device for ultrasonic treatment, which can be implanted into the human body, enable the treatment to be performed directly on the site to be treated, and allow for regular treatment without the need to travel to the hospital frequently.

### BRIEF SUMMARY OF THE INVENTION

The present invention aims to solve the aforementioned problems and deficiencies by providing an elastic mesh sleeve device for ultrasonic treatment. The elastic mesh sleeve device for ultrasonic treatment can be safely used in a human body due to the biomaterials that it is made of, and thus can directly sleeve a site to be treated inside a body of a user to perform ultrasonic therapy on that site, so that ultrasonic waves can directly reach the site to be treated to ensure that cells of pathological tissues can be killed, and since the ultrasonic waves do not need to pass through the skin, the impact of the ultrasonic waves on the skin is significantly reduced. Moreover, a micro ultrasonic vibration module does not only perform ultrasonic vibration, but also has a wireless charging function and a wireless control function. The elastic mesh sleeve device can be used for a long time inside the human body once it is implanted into the human body, so that the patient can receive regular ultrasonic therapy at home without the need to travel to the hospital frequently, and the costs in terms of both time and money are greatly reduced.

The present invention is achieved as follows:

An elastic mesh sleeve device for ultrasonic treatment, comprising an elastic biomaterial mesh sleeve and a micro ultrasonic vibration module mounted on the elastic biomaterial mesh sleeve; the micro ultrasonic vibration module comprises a housing and a housing cover assembled to each other, wherein an accommodating cavity is defined between an enclosure formed by the housing and the housing cover; the micro ultrasonic vibration module also comprises an ultrasonic vibration assembly and a driving control assembly, both disposed in the accommodating cavity; the ultrasonic vibration assembly comprises a slidable block, resilient tabs disposed at two ends of the slidable block respectively, and at least one driving coil; at least one permanent magnet block is provided on the slidable block; the resilient tabs provides buffering and bouncing functions against the slidable block as the slidable block slides; said at least one driving coil cooperates with said at least one permanent magnet block to generate a magnetic driving effect; the driving control assembly comprises a circuit board on which a wireless communication module is disposed, and a battery and a wireless charging coil which are electrically connected to the circuit board; said at least one driving coil is electrically connected to the circuit board to drive the ultrasonic vibration assembly to work.

Preferably, the accommodating cavity is provided with a partition plate that partitions the accommodating cavity into a first working chamber and a second working chamber; the ultrasonic vibration assembly is disposed in the first working chamber, and the driving control assembly is disposed in the second working chamber.

Preferably, the second working chamber is stacked on the first working chamber.

Preferably, said at least one driving coil is disposed on the partition plate, and the slidable block is disposed on the housing cover.

Preferably, sensors electrically connected to the circuit board are further disposed at two ends of the housing respectively.

Preferably, transitional inclined planes are further provided on peripheral sides of the housing.

Preferably, the elastic biomaterial mesh sleeve is an integrally formed cylindrical elastic mesh sleeve.

Preferably, the elastic biomaterial mesh sleeve is a cylindrical elastic mesh sleeve with a notch;
an adhesive tape is provided extending from one end of the notch, and the adhesive tape is fixed to another end of the notch by adhesion; alternatively,
a fastening strip having a length longer than a circumferential perimeter of the elastic biomaterial mesh sleeve is disposed around the elastic biomaterial mesh sleeve.

Preferably, the elastic biomaterial mesh sleeve has dual layers, and the micro ultrasonic vibration module is sandwiched between the dual layers.

Preferably, the elastic biomaterial mesh sleeve comprises an elastic meshed outer sleeve and an elastic cover sheet attached to an inner wall of the elastic meshed outer sleeve; the micro ultrasonic vibration module is sandwiched between the elastic cover sheet and the elastic meshed outer sleeve.

The present invention has the following beneficial effects: because an elastic biomaterial mesh sleeve is used where a micro ultrasonic vibration module is nested in the elastic biomaterial mesh sleeve, the elastic mesh sleeve device can be implanted into the human body due to the biomaterials which it is made of, and after implantation, it has good biocompatibility, causes no foreign sensation, and does not result in adverse reactions, and thus can be safely used inside the human body. With such a structural design, the elastic mesh sleeve device can directly sleeve onto a site to be treated to directly perform ultrasonic therapy on that site, so that ultrasonic energy can directly reach the site to ensure that the ultrasonic energy is not weakened and cells of pathological tissues are killed. In addition, the ultrasonic waves do not need to pass through the human skin, which can greatly reduce the impact of the ultrasonic waves on the human skin. Moreover, by utilizing the ultrasonic cavitation effect generated by the ultrasonic waves, the elastic mesh sleeve device can also promote blood circulation, relieve soft tissues, reduce swelling, eliminate inflammation, improve the nutritional status of tissues, ablate a thrombus, and prevent the occurrence of thrombosis, thereby achieving an effect of assisting in self-repair of inner walls of blood vessels. Also, the elastic biomaterial mesh sleeve will not obstruct the normal functioning of the organs where the site to be treated is located due to its very good elasticity and air permeability, and its use is therefore reliable. Further, the micro ultrasonic vibration module of the present invention is provided with a driving control assembly, which does not only drive an ultrasonic vibration assembly to work, but also has a wireless charging function and a wireless control function. In this way, the elastic mesh sleeve device can be used for a long time inside the human body once being implanted, and a professional medical personnel can set parameters in a circuit board such as the intensity and duration of the ultrasonic energy being used, so that a user can receive regular ultrasonic therapy at home without the need to travel to the hospital frequently, and the costs in terms of both time and money are greatly reduced. Besides, drugs intake of the user can be greatly reduced due to the use of the present invention, and hence side effects of the drugs harmful to the body are greatly reduced, and the user's life may therefore by prolonged.

### BRIEF DESCRIPTION OF THE DRAWINGS

FIG. 1 is a schematic overall structural view of the present invention;

FIG. 2 is a schematic cross-sectional view of the present invention when the elastic biomaterial mesh sleeve has dual layers;

FIG. 3 is a schematic cross-sectional view of the present invention when the elastic biomaterial mesh sleeve has only a single layer;

FIG. 4 is a schematic diagram of a structure of a first variation of the present invention;

FIG. 5 is a schematic diagram of a structure of a second variation of the present invention;

FIG. 6 is a schematic perspective view of a micro ultrasonic vibration module of the present invention;

FIG. 7 is a first exploded structural view of a micro ultrasonic vibration module of the present invention;

FIG. 8 is a second exploded structural view of a micro ultrasonic vibration module of the present invention, where the housing cover is omitted from illustration;

FIG. 9 is a schematic cross-sectional view of a housing, a partition plate, and a housing cover of the present invention;

FIG. 10 is a schematic diagram of a use state of the present invention when charging; and

FIG. 11 is a schematic diagram of a structure of a third variation of the present invention.

### DETAILED DESCRIPTION OF THE INVENTION

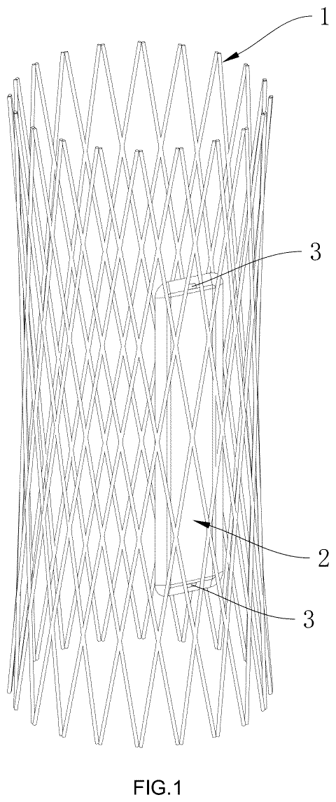

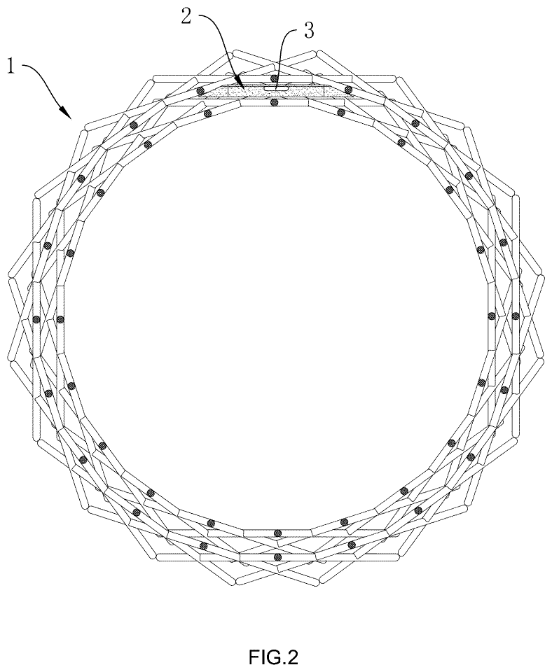

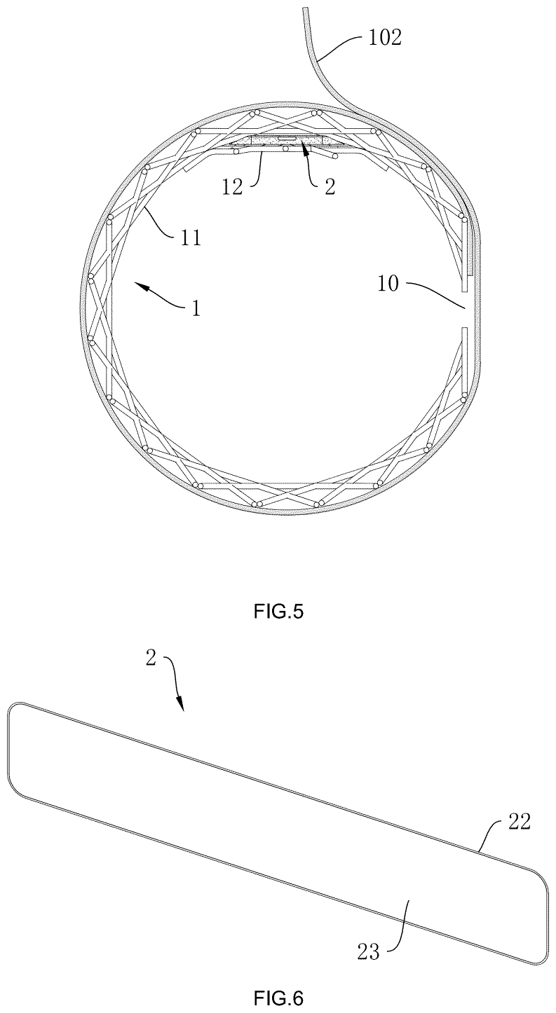

As shown in FIG. 1 and FIG. 2, the elastic mesh sleeve device for ultrasonic treatment of the present invention comprises an elastic biomaterial mesh sleeve 1 and a micro ultrasonic vibration module 2 mounted on the elastic biomaterial mesh sleeve 1. As shown in FIG. 6 to FIG. 8, the micro ultrasonic vibration module 2 comprises a housing 22 and a housing cover 23 assembled to each other, wherein an accommodating cavity 21 is defined between an enclosure formed by the housing 22 and the housing cover 23; the micro ultrasonic vibration module 2 also comprises an ultrasonic vibration assembly 24 and a driving control assembly 25, both disposed in the accommodating cavity 21. The ultrasonic vibration assembly 24 comprises a slidable block 242, resilient tabs 243 disposed at two ends of the slidable block 242 respectively, and at least one driving coil 244; at least one permanent magnet block 241 is provided on the slidable block 242; the resilient tabs 243 provides buffering and bouncing functions against the slidable block 242 as the slidable block 242 slides; said at least one driving coil 244 cooperates with said at least one permanent magnet block 241 to generate a magnetic driving effect. The driving control assembly 25 comprises a circuit board 252 on which a wireless communication module 251 is disposed, and a battery 253 and a wireless charging coil 254 which are electrically connected to the circuit board 252. Said at least one driving coil 244 is electrically connected to the circuit board 252 to drive the ultrasonic vibration assembly 24 to work.

The working principle of the ultrasonic vibration assembly 24 is as follows: The battery 253 supplies power to said at least one driving coil 244 through the circuit board 252, and a current flows into said at least one driving coil 244 to generate a magnetic field, which drives said at least one permanent magnet block 241 and thus the slidable block 242 to reciprocate, thereby generating high-frequency vibrations, which propagate through a medium such as gas, liquid, and solid to form ultrasonic waves. Compared with conventional eccentric motor, the ultrasonic vibration assembly 24 has the advantage of reducing, if not eliminating, motion inertia, and can therefore realize rapid start/stop of the reciprocation of the slidable block 242; further, the ultrasonic vibration assembly 24 does not require any space for eccentric rotation as in the case of using eccentric motor in the prior art, as a result, an overall thickness of the micro ultrasonic vibration module 2 can be smaller. Apart from using the aforementioned ultrasonic vibration assembly 24 as described above, the present invention can be alternatively implemented by using an ultrasonic transducer or an ultrasonic vibration motor in lieu of the ultrasonic vibration assembly 24. However, product performance is slightly poorer if the ultrasonic vibration motor is used.

Moreover, by providing the wireless communication module 251, a user can send on/off signals to the wireless communication module 251 through a wireless communication device, so that the circuit board 252 is remotely driven to control the start/stop of the ultrasonic vibration assembly 24. The wireless communication module 251 is an ultra-micro Bluetooth® communication module, WiFi® communication module, or the like. By utilizing a wireless communication module 251 to be linked to a device like a computer, a smartphone, or a tablet computer, remote operations, monitoring, and data transmissions of the micro ultrasonic vibration module 2 are realized.

In practical application, said at least one driving coil 244, the wireless charging coil 254, and the battery 253 are all electrically connected to the circuit board 252 through conducting wires, and the wireless communication module 251 is built-in to the circuit board 252.

Specifically, the accommodating cavity 21 may be a recess on the housing 22, or may be a recess on the housing cover 23.

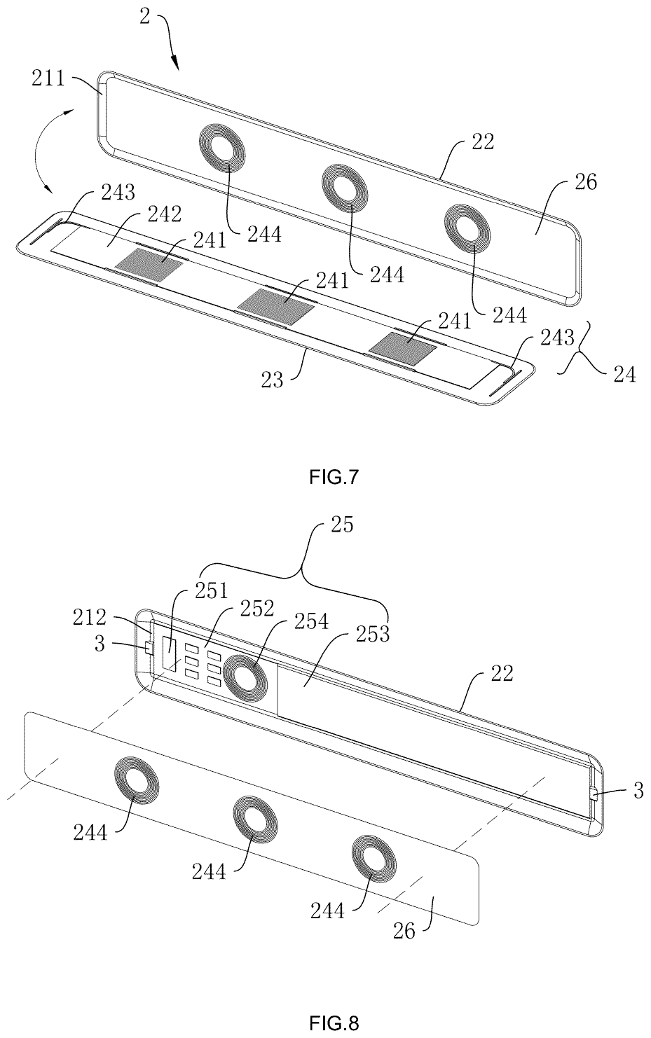

As shown in FIG. 7 to FIG. 9, the accommodating cavity 21 is provided with a partition plate 26 for partitioning the accommodating cavity 21 into a first working chamber 211 and a second working chamber 212. The ultrasonic vibration assembly 24 is disposed in the first working chamber 211, and the driving control assembly 25 is disposed in the second working chamber 212. By partitioning the accommodating cavity 21 into two independent chambers, the ultrasonic vibration assembly 24 and the driving control assembly 25 can be prevented from interfering with each other, so that the operations of the two are more stable and reliable.

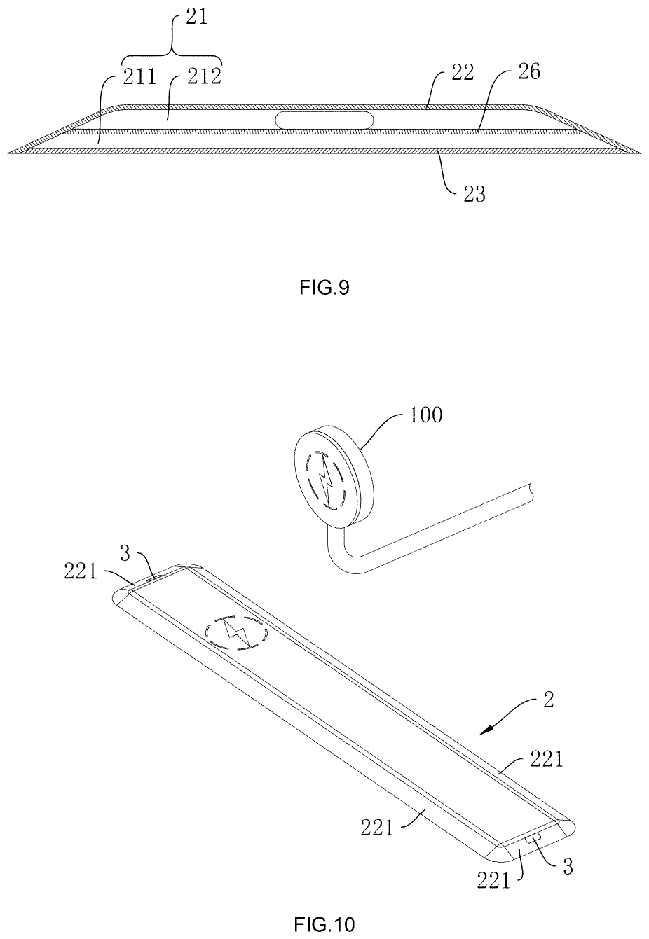

As shown in FIG. 9, the second working chamber 212 is stacked on the first working chamber 211. This enables the ultrasonic vibration assembly 24 to be disposed along a lengthwise direction of the micro ultrasonic vibration module 2, so that the vibration frequencies of various parts of the micro ultrasonic vibration module 2 are more balanced, and so the ultrasonic vibration treatment effect is better. Moreover, by positioning the second working chamber 212 above the first working chamber 211, the wireless charging coil 254 is positioned closer to a top surface of the housing 22, so that the wireless charging coil 254, as a receiving end of an external wireless charging device, can be closer to a transmitting end 100 of the external wireless charging device during wireless charging, thereby enabling the wireless transmission of electric energy to be more stable, reliable, and difficult to interrupt.

As shown in FIG. 7, said at least one driving coil 244 is disposed on the partition plate 26, and the slidable block 242 is disposed on the housing cover 23. In this way, it is unnecessary to move the conducting wires out onto the housing cover 23, thereby facilitating assembly and preventing the conducting wires from affecting the sliding of the slidable block 242.

As shown in FIGS. 8 and 10, sensors 3 electrically connected to the circuit board 252 are further disposed at two ends of the housing 22 respectively. Each of the sensors 3 can be a heart rate detection sensor, a blood oxygen detection sensor, a body temperature detection sensor, an image sensor mounted with a micro camera, or the like. In this way, it is convenient to observe and detect the health condition of the user, thereby increasing the usage functions of the elastic mesh sleeve device. When using two image sensors mounted with two micro cameras, mesh holes of the elastic biomaterial mesh sleeve 1 will not block the vision of the two micro cameras, thereby providing reliable use.

As shown in FIG. 10, transitional inclined planes 221 are further provided on peripheral sides of the housing 22. In this way, after the micro ultrasonic vibration module 2 is wrapped, excessively sharp edges and corners will not be formed on the elastic biomaterial mesh sleeve 1, so that after the elastic mesh sleeve device is implanted into the human body, no obvious discomfort will be caused.

As shown in FIG. 1, the elastic biomaterial mesh sleeve 1 is an integrally formed cylindrical elastic mesh sleeve. In this way, the elastic biomaterial mesh sleeve 1 is easier to produce and manufacture, and has a simpler structure.

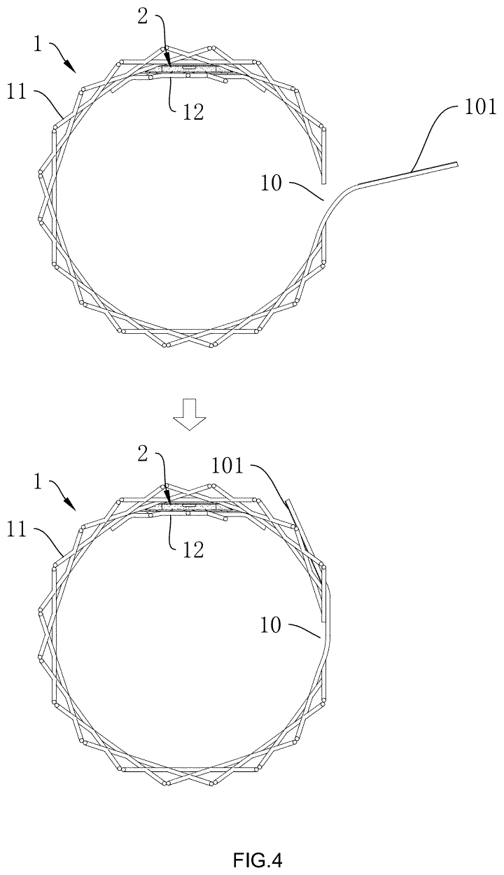

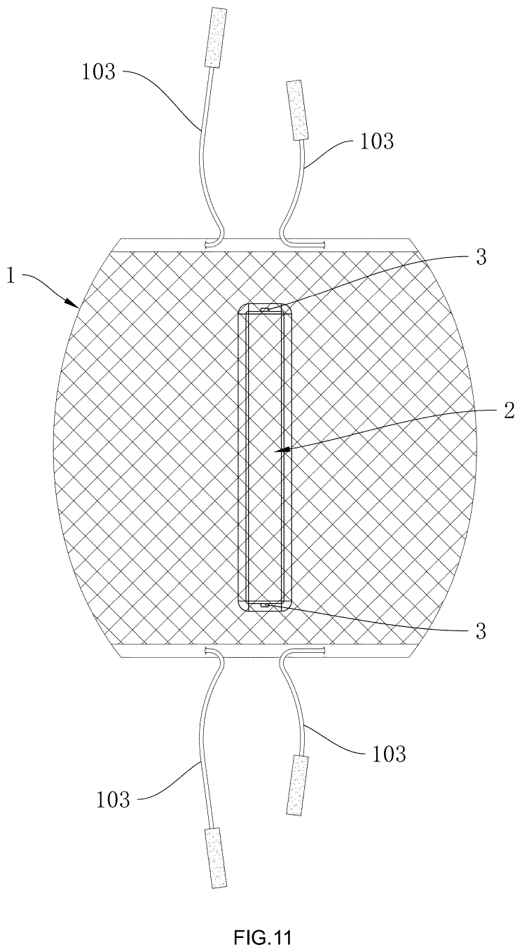

As shown in FIG. 4 and FIG. 5, the elastic biomaterial mesh sleeve 1 is a cylindrical elastic mesh sleeve with a notch 10, in other words, a radial cross-section of the elastic biomaterial mesh sleeve 1 is in a C shape. In this way, a size of an internal space defined by the substantial enclosure of the elastic biomaterial mesh sleeve 1 can be adjusted to fit with different sizes of different sites which the elastic biomaterial mesh sleeve 1 sleeves during treatment. When the size of the internal space of the elastic biomaterial mesh sleeve 1 needs to be reduced, the following solution can be adopted: an adhesive tape 101 is provided extending from one end of the notch 10, and the adhesive tape 101 is fixed to another end of the notch 10 by adhesion, as shown in FIG. 4. When the size of the internal space of the elastic biomaterial mesh sleeve 1 needs to be increased, the following solution can be adopted: a fastening strip 102 having a length longer than a circumferential perimeter of the elastic biomaterial mesh sleeve 1 is disposed around the elastic biomaterial mesh sleeve 1, and two ends of the fastening strip 102 are fixed by adhesion through biological glue or by being tied into a knot, as shown in FIG. 5. Alternatively, as shown in FIG. 11, top and bottom ends of the elastic biomaterial mesh sleeve 1 are each provided with a tie string 103 disposed at least circumferentially around the elastic biomaterial mesh sleeve 1, so that sizes of the top and bottom ends of the elastic biomaterial mesh sleeve 1 can be conveniently controlled by tightening or loosening the tie strings 103.

In order to further improve the structure of the elastic biomaterial mesh sleeve 1, as shown in FIG. 2, the elastic biomaterial mesh sleeve 1 has dual layers, and the micro ultrasonic vibration module 2 is sandwiched between the dual layers. The micro ultrasonic vibration module 2 can be conveniently nested into the elastic biomaterial mesh sleeve 1 due to the dual layer structure.

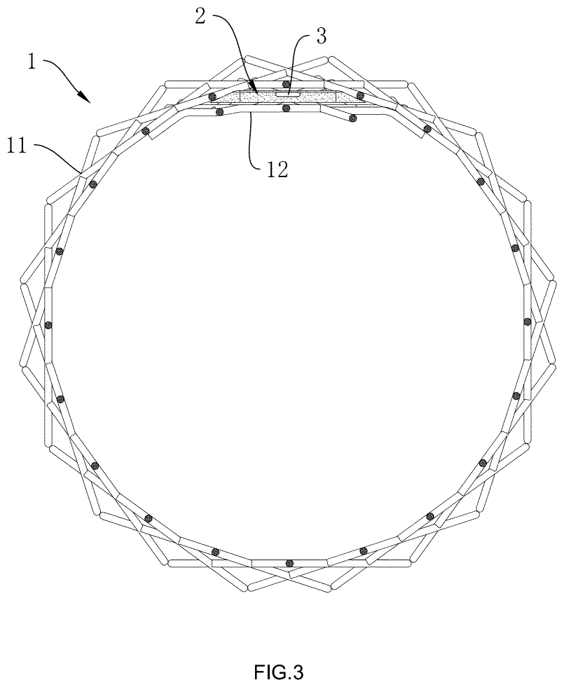

As shown in FIG. 3, the micro ultrasonic vibration module 2 can also be mounted to the elastic biomaterial mesh sleeve 1 as follows: the elastic biomaterial mesh sleeve 1 comprises an elastic meshed outer sleeve 11 and an elastic cover sheet 12 attached to an inner wall of the elastic meshed outer sleeve 11, and the micro ultrasonic vibration module 2 is sandwiched between the elastic cover sheet 12 and the elastic meshed outer sleeve 11. Specifically, the elastic cover sheet 12 can be fixed on the inner wall of the elastic meshed outer sleeve 11 by biomaterial glue or biomaterial suture.

The elastic biomaterial mesh sleeve 1, the elastic meshed outer sleeve 11 and the elastic cover sheet 12 described above can be made by using biomaterial films with good air permeability, such as protein-based electronic skin PBES, or a silk fibroin-polyurethane SF-PU composite film, or a silk fibroin composite film SFCM, and the mesh holes are formed thereon. The commonly known and used WAB® biological adhesive manufactured by Jinan Shiji Bioadhesive Research Institute can be used as said biomaterial glue. The biomaterial suture can be made of polyester, polypropylene, catgut, poly(p-dioxanone), polylactic acid, polycaprolactone or other materials with good biocompatibility.

What is claimed is:

1. An elastic mesh sleeve device for ultrasonic treatment, comprising an elastic biomaterial mesh sleeve and a micro ultrasonic vibration module mounted on the elastic biomaterial mesh sleeve;
the micro ultrasonic vibration module comprises a housing and a housing cover assembled to each other, wherein an accommodating cavity is defined between an enclosure formed by the housing and the housing cover;
the micro ultrasonic vibration module also comprises an ultrasonic vibration assembly and a driving control assembly, both disposed in the accommodating cavity;
the ultrasonic vibration assembly comprises a slidable block, resilient tabs disposed at two ends of the slidable block respectively, and at least one driving coil; at least one permanent magnet block is provided on the slidable block; the resilient tabs provides buffering and bouncing functions against the slidable block as the slidable block slides; said at least one driving coil cooperates with said at least one permanent magnet block to generate a magnetic driving effect;
the driving control assembly comprises a circuit board on which a wireless communication module is disposed, and a battery and a wireless charging coil which are electrically connected to the circuit board; said at least one driving coil is electrically connected to the circuit board to drive the ultrasonic vibration assembly to work.

2. The elastic mesh sleeve device of claim 1, wherein the accommodating cavity is provided with a partition plate that partitions the accommodating cavity into a first working chamber and a second working chamber; the ultrasonic vibration assembly is disposed in the first working chamber, and the driving control assembly is disposed in the second working chamber.

3. The elastic mesh sleeve device of claim 2, wherein the second working chamber is stacked on the first working chamber.

4. The elastic mesh sleeve device of claim 3, wherein said at least one driving coil is disposed on the partition plate, and the slidable block is disposed on the housing cover.

5. The elastic mesh sleeve device of claim 1, wherein sensors electrically connected to the circuit board are further disposed at two ends of the housing respectively.

6. The elastic mesh sleeve device of claim 1, wherein transitional inclined planes are further provided on peripheral sides of the housing.

7. The elastic mesh sleeve device of claim 1, wherein the elastic biomaterial mesh sleeve is an integrally formed cylindrical elastic mesh sleeve.

8. The elastic mesh sleeve device of claim 1, wherein the elastic biomaterial mesh sleeve is a cylindrical elastic mesh sleeve with a notch;
an adhesive tape is provided extending from one end of the notch, and the adhesive tape is fixed to another end of the notch by adhesion; alternatively,
a fastening strip having a length longer than a circumferential perimeter of the elastic biomaterial mesh sleeve is disposed around the elastic biomaterial mesh sleeve.

9. The elastic mesh sleeve device of claim 1, wherein the elastic biomaterial mesh sleeve has dual layers, and the micro ultrasonic vibration module is sandwiched between the dual layers.

10. The elastic mesh sleeve device of claim 1, wherein the elastic biomaterial mesh sleeve comprises an elastic meshed outer sleeve and an elastic cover sheet attached to an inner wall of the elastic meshed outer sleeve; the micro ultrasonic vibration module is sandwiched between the elastic cover sheet and the elastic meshed outer sleeve.
